(19) United States
(12) Patent Application Publication
(10) Pub. No.: US 2024/0081973 A1
(43) Pub. Date: Mar. 14, 2024

(54) Method for positioning an interpositional ophthalmological implant using ab-interno approach

(71) Applicant: CILIATECH, Chavanod (FR)

(72) Inventors: Olivier BENOIT, EPAGNY METZ-TESSY (FR); Philippe SOURDILLE, GUERANDE (FR)

(21) Appl. No.: 18/243,938

(22) Filed: Sep. 8, 2023

(30) Foreign Application Priority Data
Sep. 13, 2022 (FR) 2209203

(51) Int. Cl. A61F 2/14

(52) U.S. Cl. CPC A61F 2/142

(57) ABSTRACT

A method is provided for inserting an ophthalmological implant between a sclera and a uveal tissue in an eye of a patient. The method includes providing an ophthalmological implant having an anterior edge intended to face the anterior chamber and an posterior edge opposite the anterior edge, and making at least one opening in a corneal tissue. An opening is made in the iris root to form an insertion channel and the ophthalmological implant is inserted through the insertion channel. The ophthalmological implant is positioned between a sclera and a uveal tissue at a position, where the anterior edge of the ophthalmological implant is located in or beyond the insertion channel. The insertion channel is closed so that an iris tissue is disposed between the anterior edge and an anterior chamber of the eye of the patient.

### RELATED APPLICATION

This application claims the benefit of priority from French Patent Application No. 22 09203, filed on Sep. 13, 2023, the entirety of which is incorporated by reference.

### FIELD OF THE INVENTION

The invention relates to an interpositional ophthalmological implant intended to keep the sclera and the ciliary body permanently separated from one another in order to lower intraOcular pressure (IOP).

### DESCRIPTION OF THE RELATED ART

A human eye is usually split in two distinct anatomical volumes:
the anterior segment which contains the lens, the cornea, the ciliary body and the iris, and in between the corneal and the lens a space named “anterior chamber”,
the posterior segment with the vitreous and the retina.

The conjunctiva and the sclera (the white non-transparent part or the eye) covers mainly the posterior segment.

Glaucoma is a sight threatening disorder characterised by chronic IntraOcular Pressure (“IOP”) increase beyond the normal limits. The reason for this IOP increase lies—very schematically—in the difficulty for the aqueous humour to drain out of the anterior chamber.

Two ways of aqueous humour circulation are well known: the trabecular and the uveo-scleral outflows. Both of them—alone or in combination—can be the cause for the IOP increase, and both of them can be purpose for treating the disease.

Surgery is one way to get IOP under control. Several techniques exist, and they all consist into re-establishing a sustainable and controlled drainage of the aqueous humour from the AC to either the sub-conjunctival or the supraciliary/suprachoroidal spaces. This is obtained by incising tissues and creating artificial egresses, with two approaches:
ab-externo: the surgery is performed from the outside of the eye, the conjunctiva, sclera, corneal and other tissues may be incised or removed, or
ab-interno: the surgery is performed from the inside of the eye, specifically from the anterior chamber; an incision is performed in the cornea and the surgeon manipulates surgical tools from the inside of the anterior chamber.

Surgeries can be performed with or without implant. Implants are expected to prolong the IOP-lowering effect of the surgery by keeping openings and artificially created channels or aqueous egresses open and functional.

In the case of ab-interno approach, it exists a specific technique called “cyclodialysis”, which consists in making a local cut in the iris root, at the level of the scleral spur: the anterior chamber is then in direct contact with the supraciliary/suprachoroidal space and this enhances the uveo-scleral outflow. It is known by long experience this technique can be efficient for decreasing IOP, however results are limited in time as tissues are healing and the artificial opening closes naturally in the weeks following the surgery. In such case, implants help to keep the artificial openings stable and can maintain surgery benefits on IOP for several months or years: to do so, these so-called cyclodialysis implants must form a conduit between the anterior chamber and the supraciliary/suprachoroidal space. Generally, these implants have the shape of a tube or a plate, with or without lumens and channels, porous or not, but always with an anterior or “inflow” and a posterior or “outflow” ends or portions, and are positioned with the anterior end or inflow portion in the anterior chamber and the posterior end or outflow portion in the supraciliary or suprachoroidal space.

The so-called iris root is the place where the ciliary muscle holds on the scleral spur, it is a piece of fibrous tissue of approx. 500 μm thick.

Cyclodialysis consists into making the anterior chamber and the sub-scleral space to communicate directly, the iris root tissues are cut/removed and are no longer a physical obstacle. In cyclodialysis surgery without implant, the effect on IOP disappears once the tissues are reformed after scarring. In line with this information, it is common belief that if a physical obstacle would appear at the anterior end of a cyclodialysis implant, this one would not drain aqueous humour and would not help to reduce IOP. In other words: the anterior end of a cyclodialysis drainage implant shall not be positioned posterior to the iris root tissues (see for example US2019/0038462 A1). For the cyclodialysis drainage implants which are positioned with an ab-interno approach, this means they shall not be inserted too posterior into the sub-scleral space

However, natural anatomy varies from individuals, and today's insertion techniques cannot warranty the repeatability of a perfect positioning of the glaucoma drainage device from one patient to the other. To ascertain the anterior end of a cyclodialysis drainage implant is always inside the anterior chamber, manufacturers design their implant and insertion tool for, and recommend a portion of the body of the cyclodialysis drainage implant remains visible in the anterior chamber after insertion.

It is known by literature that any implant or part of the body of an implant which stays in the anterior chamber and close to (not even in contact) the cornea for a long period of time can cause Endothelial Cells Loss (“ECL”), which can turn in dramatic sight threatening complications for the eye, with ultimate curative action being removal of the device and exogeneous corneal graft. This is the case of implants for cyclodialysis: once in their final location; the anterior end of such implants protrudes permanently in the anterior chamber at the place where the scleral spur joins the cornea.

Although this risk is well identified, surgeons continue to use such devices as they believe that their efficiency is necessarily conditioned by keeping the artificial opening at the iris root open. However, the risk coming with and the manual skills necessary for placing this type of glaucoma drainage device limits its usage, esp. in patient showing low endothelial cells count prior to surgery.

Ab-externo incisional surgeries can be efficient, but they are usually more invasive and show more morbidities than ab-interno ones.

A need exists for a safer method for placing a drainage implant in the sub-scleral space, without the morbidities of the ab-externo approach, and without the ECL risk coming with regular cyclodialysis drainage implants.

### SUMMARY OF THE INVENTION

Given the foregoing, the present invention proposes a method for inserting an ophthalmological implant between a sclera and a uveal tissue in an eye of a patient, comprising:
providing an ophthalmological implant having an anterior edge intended to face the anterior chamber and an posterior edge opposite the anterior edge,
making at least one opening in a corneal tissue,
opening the iris root to form an insertion channel,
inserting the ophthalmological implant through the insertion channel,
positioning the ophthalmological implant between a sclera and a uveal tissue at a position wherein the anterior edge of the ophthalmological implant is located in or beyond the insertion channel,
closing the insertion channel so that an iris tissue is disposed between the anterior edge and an anterior chamber of the eye of the patient.

The ophthalmic implant is thus positioned with its anterior edge which does not protrude in the anterior chamber. The anterior edge is hence embedded inside the iris-root tissues, or posterior to it.

As previously mentioned, it is common belief that if a physical obstacle would appear at the anterior end of a cyclodialysis implant, this one would not drain aqueous humour and would not help to reduce IOP.

Two clinical studies have been performed and surprisingly showed that IOP can be significantly reduced even with an implant disposed behind the iris root.

In these clinical studies, an ophthalmological implant was implanted into 42 patients with the implant positioned between the sclera and the uveal tissue with the iris root closed (i.e. without any artificial channel).

FIG. 1 shows the blended mean IOP variation in percentage of the patients during 12 months. “Preop” refers to the IOP value before the implantation of the implant, “D1” refers to Day 1 and “M3” refers to Month 3 up to Month 12 (“M12”). Results clearly show that IOP has been reduced by more than 30% along the 12 months period, and below 18 mm of mercury which is the commonly accepted threshold below which glaucoma symptoms do not progress any longer. Results also showed that pharmacological treatments have been significantly reduced by over 80% along the 12 months when compared to pre-operative values.

These results show that the main preconceived notion professionals had (the presence of intact iris root tissues at the implant anterior edge is an obstacle to aqueous humour circulation) is deconstructed by these two studies.

This new method thus allows to significantly reduce IOP while avoiding endothelial cells loss.

According to an embodiment, the ophthalmological implant is positioned between a sclera and a uveal tissue so that the anterior edge is not in the anterior chamber.

According to an embodiment, the method further comprises a step of inserting instruments within the at least one opening in the corneal tissue to allow the ophthalmological implant to be inserted through the at least one opening.

According to an embodiment, the iris root is opened using a cyclodialysis spatula.

According to an embodiment, the inserting step comprises the injection of a viscoelastic fluid in the anterior chamber and/or between the sclera and the uveal tissues.

According to an embodiment, the insertion channel comprises an anterior end facing the anterior chamber and a posterior end facing a space between the sclera and the uveal tissues, the anterior end of the insertion channel being closed during the closing step.

According to an embodiment, the anterior edge of the ophthalmological implant is positioned between the anterior and posterior ends of the insertion channel.

According to an embodiment, the anterior edge of the ophthalmological implant is positioned out of the insertion channel.

According to an embodiment, both the anterior and the posterior ends of the insertion channel are closed during the closing step.

According to an embodiment, the insertion channel comprises two parts which are separate from each other during the opening step, the step of closing the insertion channel comprises bringing said two parts of the insertion channel into contact with each other.

### BRIEF DESCRIPTION OF THE DRAWINGS

The invention is described in more detail below by way of the figures that show only one preferred embodiment of the invention.

FIG. 1 schematically shows a graph illustrating the blended mean IOP variation of patients during 12 months after implantation of an ophthalmic implant behind the iris root and without opening the iris root;

FIG. 2 schematically shows a first embodiment of a method wherein the ophthalmic implant is positioned in a sub-scleral position;

FIG. 3 schematically shows a second embodiment of the method wherein the ophthalmic implant is positioned inside the iris root with an anterior end of the iris root which remains closed/occluded.

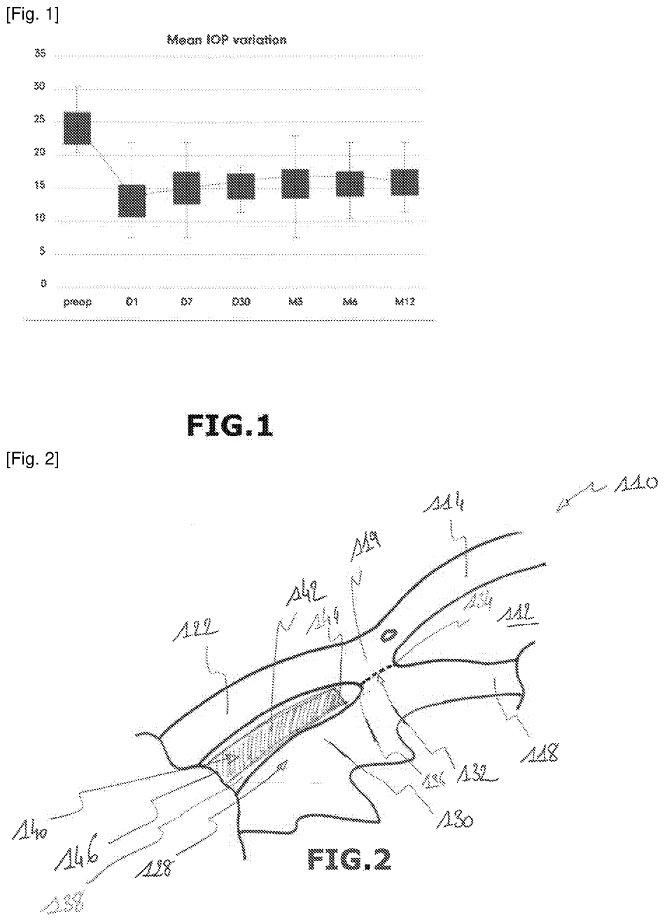

### DETAILED DESCRIPTION OF THE PREFERRED EMBODIMENTS

In the description which follows, the drawing figures are not necessarily to scale and certain features may be shown in generalized or schematic form in the interest of clarity and conciseness or for informational purposes. In addition, although making and using various embodiments are discussed in detail below, it should be appreciated that as described herein are provided many inventive concepts that may embodied in a wide variety of contexts. Embodiments discussed herein are merely representative and do not limit the scope of the invention. It will also be obvious to one skilled in the art that all the technical features that are defined relative to a process can be transposed, individually or in combination, to a device and conversely, all the technical features relative to a device can be transposed, individually or in combination, to a process.

The terms “comprise” (and any grammatical variation thereof, such as “comprises” and “comprising”), “have” (and any grammatical variation thereof, such as “has” and “having”), “contain” (and any grammatical variation thereof, such as “contains” and “containing”), and “include” (and any grammatical variation thereof such as “includes” and “including”) are open-ended linking verbs. They are used to specify the presence of stated features, integers, steps or components or groups thereof, but do not preclude the presence or addition of one or more other features, integers, steps or components or groups thereof. As a result, a method, or a step in a method, that “comprises”, “has”, “contains”, or “includes” one or more steps or elements possesses those one or more steps or elements but is not limited to possessing only those one or more steps or elements.

The present invention provides a method for inserting an ophthalmological implant between a sclera and a uveal tissue in an eye of a patient. The ophthalmologic implant may be a cyclodialysis implant or a permanent interpositional ophthalmologic implant.

For sake of clarity, the words “anterior edge” will be used further in the document to represent the proximal edge of an ophthalmologic implant, i.e. the part of the body of the implant the closest to the anterior chamber. It will be used as an equivalent to proximal edge, inflow edge, proximal portion, inflow portion.

Likewise “posterior edge” will be used to represent the distal edge of the ophthalmologic implant, i.e. the part of the body of the implant the farthest to the anterior chamber. It will be used as an equivalent to distal edge, outflow edge, distal portion, outflow portion.

The word “sub-scleral space” shall be understood as the anatomical space formed by the “supraciliary space” and the “suprachoroidal space”.

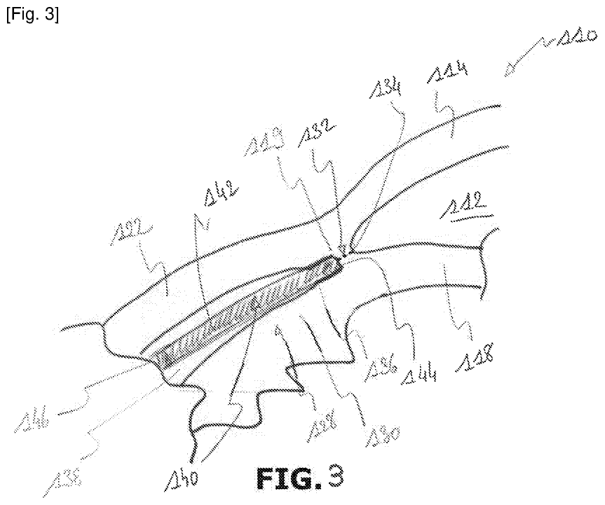

FIGS. 2 and 3 show a cross-section of a portion of an eye 110 depicting the anterior chamber 112 which is disposed between the cornea 114 and the lens (not shown) bounded at its peripheral portion by the iris 118.

The sclera 122 is connected to the periphery of the cornea 114. The sclera 122 overlies the ciliary body 128 which is connected to the iris 118 and includes the ciliary muscle 130. The iris root 119 is a part of the iris 118 which is fixed to the sclera 122, at the junction between the cornea 114 and the sclera 122.

An implant 140 is interposed between the sclera 122 and the ciliary body 128. This implant 140 may be a cyclodialysis implant or a permanent interpositional ophthalmic implant.

The implant 140 comprises a body 142 having an anterior edge 144 intended to face the anterior chamber 112 and a posterior edge 146 opposite the anterior edge 144.

The spacing produced at this location between the sclera 122 and the ciliary body 128 allows for permanent collection of aqueous humor as close as possible to the area of physiological uveo-scleral flow (the spacing effect creates an area of least resistance to aqueous humor flow). Such an implant 140 positioned in this way provides a significant gain in increasing physiological uveo-scleral outflow.

The present invention provides a method for inserting this implant 140 between the sclera 122 and a uveal tissue, such as the ciliary body 128, in an eye 110 of a patient.

At least one opening is made in a corneal tissue of the cornea 114. Instruments may be inserted within the at least one opening in the corneal tissue to allow the implant 140 to be inserted through the at least one opening. Instruments are then removed from the eye when the implant 140 is positioned.

A gonioscopic lens may be used by the surgeon to perform the following steps.

The iris root 119 is then opened to form an insertion channel 132. The iris root 119 may be opened using a cyclodialysis spatula. The iris root 119 is preferably peeled off from the sclera 122 to form the insertion channel 132.

The implant 140 is then inserted through the insertion channel 132. The implant 140 is positioned between the sclera 122 and a uveal tissue at a position wherein the anterior edge 144 of the implant 140 is located in or beyond the insertion channel 132. FIG. 2 shows the implant 140 located beyond the insertion channel 132 according to a first embodiment and FIG. 3 shows the implant located in the insertion channel 132 according to a second embodiment.

The insertion channel 132 is then closed so that an iris tissue is disposed between the anterior edge 144 and the anterior chamber 112 of the eye of the patient. In other words, at the end of the positioning step, the implant 140 is positioned between the sclera 122 and a uveal tissue so that the anterior edge 144 is not in the anterior chamber 112. This last step can be realized by the surgeon, or the tissues “self-close” if the opening of the iris root is small enough.

The insertion channel 132 comprises an anterior end 134 facing the anterior chamber 112 and a posterior end 136 facing a space 138 between the sclera 122 and the uveal tissues. What is meant by “an iris tissue is disposed between the anterior edge 144 and the anterior chamber 112” is that at least the anterior end 134 of the insertion channel 132 is closed during the closing step. Hence, the two tissue parts which are separate from each other to form the insertion channel 132 when the iris root 119 is opened are then brought into contact with each other. The tissues then heal and the artificial opening closes naturally in the weeks following the surgery.

According to the first embodiment of FIG. 2, the anterior edge 144 of the implant 140 is positioned beyond or out of the insertion channel 132 after the positioning step. Both the anterior 134 and posterior 136 ends of the insertion channel 132 are then closed. In other words, the two tissue parts which are separate from each other to form the insertion channel 132 when the iris root 119 is opened are then brought into contact with each other at both the anterior 134 and posterior 136 ends of the insertion channel 132.

According to the second embodiment of FIG. 3, the anterior edge 144 of the implant 140 is positioned in the insertion channel 132 after the positioning step. In other words, the anterior edge 144 of the implant 140 is positioned between the anterior 134 and posterior 136 ends of the insertion channel 132 after the positioning step. The anterior edge 144 is for example disposed at mid-root, i.e. near the middle of the iris root 119 or the insertion channel 132. The anterior 134 end of the insertion channel 132 is then closed. In other words, the two tissue parts which are separate from each other to form the insertion channel 132 when the iris root 119 is opened are then brought into contact with each other at the anterior 134 136 end of the insertion channel 132.

A viscoelastic fluid may also be injected in the anterior chamber 112 and/or between the sclera 122 and the uveal tissues to help positioning the implant 140.

1. A method for inserting an ophthalmological implant between a sclera and a uveal tissue in an eye of a patient, comprising:
providing an ophthalmological implant having an anterior edge intended to face the anterior chamber and an posterior edge opposite the anterior edge,
making at least one opening in a corneal tissue,
opening the iris root to form an insertion channel,
inserting the ophthalmological implant through the insertion channel,
positioning the ophthalmological implant between a sclera and a uveal tissue at a position wherein the anterior edge of the ophthalmological implant is located in or beyond the insertion channel, and
closing the insertion channel so that an iris tissue is disposed between the anterior edge and an anterior chamber of the eye of the patient.

2. The method according to claim 1, wherein the ophthalmological is positioned between a sclera and a uveal tissue so that the anterior edge is not in the anterior chamber.

3. The method according to claim 1, further comprising a step of inserting instruments within the at least one opening in the corneal tissue to allow the ophthalmological implant to be inserted through the at least one opening.

4. The method according to claim 1, wherein the iris root is opened using a cyclodialysis spatula.

5. The method according to claim 1, wherein the inserting step comprises the injection of a viscoelastic fluid in the anterior chamber and/or between the sclera and the uveal tissues.

6. The method according to claim 1, wherein the insertion channel comprises an anterior end facing the anterior chamber and a posterior end facing a space between the sclera and the uveal tissues, the anterior end of the insertion channel being closed during the closing step.

7. The method according to claim 6, wherein the anterior edge of the ophthalmological implant is positioned between the anterior and posterior ends of the insertion channel.

8. The method according to claim 6, wherein the anterior edge of the ophthalmological implant is positioned out of the insertion channel.

9. The method according to claim 8, wherein both the anterior and the posterior ends of the insertion channel are closed during the closing step.

10. The method according to claim 1, wherein the insertion channel comprises two parts which are separate from each other during the opening step, the step of closing the insertion channel comprises bringing said two parts of the insertion channel into contact with each other.
